(19) United States
(12) Patent Application Publication
(10) Pub. No.: US 2018/0325788 A1
(43) Pub. Date: Nov. 15, 2018

(54) PHOTOCURABLE COMPOSITION TO BE USED ON FINGERNAILS OR ARTIFICIAL NAILS, BASE COAT AGENT CONTAINING SAME, CURED ARTICLE THEREOF, METHOD FOR PRODUCING CURED ARTICLE THEREOF, METHOD FOR DETACHING CURED ARTICLE THEREOF, METHOD FOR COATING BY USING SAME, AND METHOD FOR USING SAME

(71) Applicant: THREE BOND CO., LTD., Tokyo (JP)

(72) Inventor: Koichi TAKEMOTO, Tokyo (JP)

(73) Assignee: THREE BOND CO., LTD., Tokyo (JP)

(21) Appl. No.: 15/774,411

(22) PCT Filed: Oct. 26, 2016
(86) PCT No.: PCT/JP2016/081779

§ 371 Date: May 8, 2018

(30) Foreign Application Priority Data
Nov. 9, 2015 (JP) 2015-219338

(51) Int. Cl. A61K 8/368; A61K 8/35; A61Q 3/02

(52) U.S. Cl. CPC A61K 8/368; A61K 8/55; A61Q 3/02; A61K 8/35

(57) ABSTRACT

A photocurable composition for a nail or artificial nail which exhibits surface curability corresponding to a UV irradiator and LED irradiation and of which the cured product can exhibit both durability to maintain stable adhesive property in everyday life and soak off property, is colorless and transparent, and is not turbid, or a base coat agent containing the same. The photocurable composition for nail or artificial nail can contain a compound having a (meth)acryloyl group, rosin or a rosin derivative, and a photoinitiator.

### TECHNICAL FIELD

The present invention relates to a photocurable composition for nail or artificial nail, a base coat agent containing the same, a cured product of the same, a method of producing a cured product of the same, a peeling method of a cured product of the same, a covering method using the same, and a method of using the same.

### BACKGROUND ART

In the field of nails, the following operations are generally performed in order to peel off (soak off) the base coat which has already been applied. First, the top coat and the color coat are removed through sanding using a file or the like. Subsequently, cotton impregnated with acetone is wound around the nail and left in a state of being wrapped with an aluminum foil. Thereafter, the base coat not firmly fixed is lightly scraped off as the base coat becomes a state where it is not firmly fixed after a certain period of time elapses.

For this reason, in the field of UV nail gel, both contradictory properties of maintenance of durability of the coating film against water, oil, and the like in everyday life and ease of peeling off when removing the coating film for reapplication are required as properties of the base coat agent. It is disclosed in JP 2013-183754 A that a photocurable composition (UV nail gel) for nail or artificial nail which is suitable for soak off can be provided by a technique to add a large amount of filler to a UV nail gel.

### SUMMARY OF INVENTION

However, the technique to add a large amount of filler to a UV nail gel as in the invention described in JP 2013-183754 A has a problem to decrease the transparency and further to cause turbidness in some cases although it increases the strength of the UV nail gel.

Hitherto, it has been difficult to achieve both maintenance of the appearance and durability such as adhesive force of the cured product and soak off in everyday life while maintaining the surface curability and transparency in the base coat agent of the UV nail gel as described above.

As a result of intensive investigations, the inventors of the present invention have found out that the above object can be achieved by the following respective embodiments and thus have completed the present invention.

A first embodiment of the present invention is a photocurable composition for nail or artificial nail containing the following components (A) to (C):

component (A): a compound having a (meth)acryloyl group;

component (B): rosin and/or a rosin derivative; and

component (C): a photoinitiator.

A second embodiment of the present invention is the photocurable composition for nail or artificial nail according to the first embodiment, wherein the component (A) contains a (meth)acrylate oligomer and a (meth)acrylate monomer or a (meth)acrylamide monomer.

A third embodiment of the present invention is the photocurable composition for nail or artificial nail according to the second embodiment, wherein the (meth)acrylate monomer contains a (meth)acrylate monomer having an acidic group.

A fourth embodiment of the present invention is a base coat agent including the photocurable composition for nail or artificial nail according to any one of the first to third embodiments, wherein the base coat agent can be soaked off.

A fifth embodiment of the present invention is a cured product of the photocurable composition for nail or artificial nail according to any one of the first to third embodiments or the base coat agent according to the fourth embodiment.

A sixth embodiment of the present invention is a method of producing a cured product, the method including curing the photocurable composition for nail or artificial nail according to any one of the first to third embodiments or the base coat agent according to the fourth embodiment by an energy ray.

A seventh embodiment of the present invention is a peeling method including peeling off the cured product according to the fifth embodiment or a cured product produced by the method according to the sixth embodiment which is disposed on a substrate from the substrate using warm water or a solvent.

An eighth embodiment of the present invention is a method of covering a human nail or an artificial nail, which includes applying the photocurable composition for nail or artificial nail according to any one of the first to third embodiments or the base coat agent according to the fourth embodiment on a human nail or an artificial nail and then irradiating a coating film with an energy ray for curing.

A ninth embodiment of the present invention is a use method including covering a human nail or an artificial nail by the covering method according to the eighth embodiment and then peeling off a cured product of a covering layer using warm water or a solvent.

### DESCRIPTION OF EMBODIMENTS

In the present specification, the notation “(meth)acryl” in the specific name of a compound represents “acryl” and “methacryl”, the notation “(meth)acryloyl” represents “acryloyl” and “methacryloyl”, and the notation “(meth)acrylate” represents “acrylate” and “methacrylate”, respectively.

<Photocurable Composition for Nail or Artificial Nail>

A first embodiment of the present invention is a photocurable composition for nail or artificial nail containing the following components (A) to (C):

component (A): a compound having a (meth)acryloyl group;

component (B): rosin or a rosin derivative; and

component (C): a photoinitiator.

According to the first embodiment of the present invention, it is possible to provide a photocurable composition for nail or artificial nail which exhibits surface curability corresponding to a UV irradiator and LED irradiation and of which the cured product exhibits both durability to maintain stable adhesive property in everyday life and soak off property, is colorless and transparent, and is not turbid.

It is preferable that the photocurable composition for nail or artificial nail is a photocurable composition for covering nail or artificial nail.

Hereinafter, the details of the present invention will be described. However, the present invention is not limited only to the following embodiments.

[Component (A)]

The component (A) is not particularly limited and any compound can be used as long as it is a compound having a (meth)acryloyl group. The compound having a (meth)acryloyl group may have the (meth)acryloyl group in the form of a (meth)acryloyloxy group. In addition, the component (A) may also contain a compound having one or more epoxy groups and one or more acryloyl groups in one molecule.

Specific examples of the component (A) may include a (meth)acrylate-based compound which is a compound having a (meth)acrylate structure in the molecule and a (meth)acrylamide-based compound which is a compound having a primary, secondary, tertiary, or quaternary (meth)acrylamide structure in the molecule. Here, the (meth)acrylate-based compound is preferably a (meth)acrylate monomer which is a simple substance of a (meth)acrylate and a (meth)acrylate oligomer which is an oligomer having a (meth)acrylate structure in the molecule. In addition, the (meth)acrylamide-based compound is preferably a (meth)acrylamide monomer which is a primary, secondary, tertiary, or quaternary (meth)acrylamide simple substance in the molecule.

It is preferable that the component (A) is liquid in an atmosphere at 25° C. Incidentally, the component (A) exhibits favorable compatibility with the component (B) and component (C) to be described later.

The component (A) may be used singly or two or more kinds thereof may be used concurrently.

Here, the weight average molecular weight of the (meth)acrylate oligomer is preferably 1000 or more and less than 50000. The durability is further improved while a low viscosity is maintained when the weight average molecular weight is in such a range. Incidentally, in the present specification, a value measured by gel permeation chromatography (GPC) using polystyrene as a standard substance is adopted as the weight average molecular weight. The weight average molecular weight of the (meth)acrylate oligomer is more preferably 1000 or more and 30000 or less, still more preferably 2000 or more and 10000 or less, and particularly preferably 3000 or more and 8000 or less from the same viewpoint.

In addition, the (meth)acrylate oligomer preferably has 2 or more and 6 or less (meth)acryloyl groups in one molecule. The hardness and the surface curability are further improved when the number of (meth)acryloyl groups is in such a range. The (meth)acrylate oligomer more preferably has 2 or more and 4 or less (meth)acryloyl groups in one molecule and still more preferably two (meth)acryloyl groups in one molecule from the same viewpoint.

Specific examples of the (meth)acrylate oligomer may include a (meth)acrylate oligomer having an ester bond in a moiety other than the (meth)acrylate structural moiety in the molecule (also referred to as a (meth)acrylate oligomer having an ester bond in the present specification), a (meth)acrylate oligomer having an ether bond, a (meth)acrylate oligomer having a urethane bond, and an epoxy-modified (meth)acrylate oligomer, but it is not limited thereto. In addition, specific examples of the (meth)acrylate oligomer including these may include those having a structure in which the main skeleton is composed of bisphenol A, novolak phenol, polybutadiene, polyester, polyether, and the like, but it is not limited thereto.

As a method of synthesizing the (meth)acrylate oligomer having an ester bond, for example, a method is known in which a polyol and a polycarboxylic acid form an ester bond and (meth)acrylic acid is added to the unreacted hydroxyl group, but it is not limited to this synthesis method. As the (meth)acrylate oligomer having an ester bond, a synthetic product or a commercially available product may be used, and specific examples of the commercially available product may include ARONIX (registered trademark) M-6100, M-6200, M-6250, M-6500, M-7100, M-7300K, M-8030, M-8060, M-8100, M-8530, M-8560, M-9050, and the like manufactured by TOAGOSEI CO., LTD. and UV-3500BA, UV-3520TL, UV-3200B, UV-3000B, and the like manufactured by The Nippon Synthetic Chemical Industry Co., Ltd., but it is not limited thereto.

As a method of synthesizing the (meth)acrylate oligomer having an ether bond, for example, a method is known in which acrylic acid is added to a hydroxyl group of a polyether polyol which does not have an aromatic group or a hydroxyl group of a polyether polyol which has an aromatic group such as bisphenol, but it is not limited to this synthesis method. As the (meth)acrylate oligomer having an ether bond, a synthetic product or a commercially available product may be used, and specific examples of the commercially available product may include UV-6640B, UV-6100B, UV-3700B, and the like manufactured by The Nippon Synthetic Chemical Industry Co., Ltd., LIGHT ACRYLATE (registered trademark) 3EG-A, 4EG-A, 9EG-A, 14EG-A, PTMGA-250, BP-4EA, BP-4PA, BP-10EA, and the like manufactured by KYOEISHA CHEMICAL CO., LTD., and EBECRYL (registered trademark) 3700 and the like manufactured by DAICEL-ALLNEX LTD., but it is not limited thereto.

As a method of synthesizing the (meth)acrylate oligomer having a urethane bond, for example, synthesis is known in which a urethane bond is formed by a polyol and a polyisocyanate and a compound having a hydroxyl group and a (meth)acryloyl group in the molecule or (meth)acrylic acid is added to the remaining isocyanate group, but it is not limited to this synthesis method. As the component (A), it is preferable to add a (meth)acrylate oligomer having a urethane bond from the viewpoint of improving the durability. As the (meth)acrylate oligomer having a urethane bond, a synthetic product or a commercially available product may be used, and specific examples of the commercially available product may include AH-600, AT-600, UA-306H, UF-8001G, and the like manufactured by KYOEISHA CHEMICAL CO., LTD., but it is not limited thereto.

The (meth)acrylate monomer is not particularly limited, and known ones can be used. Among these, the (meth)acrylate monomer preferably contains a monofunctional, bifunctional, or trifunctional (meth)acrylate monomer and a (meth)acrylamide monomer and more preferably contains a monofunctional (meth)acrylate monomer or a (meth)acrylamide monomer. In addition, a plurality of other monomers can be used in combination.

Specific examples of the monofunctional (meth)acrylate monomer may include lauryl (meth)acrylate, stearyl (meth)acrylate, ethyl carbitol (meth)acrylate, tetrahydrofurfuryl (meth)acrylate, caprolactone-modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, phenoxyethyl (meth)acrylate, phenoxydiethylene glycol (meth)acrylate, phenoxytetraethylene glycol (meth)acrylate, nonylphenoxyethyl (meth)acrylate, nonylphenoxytetraethylene glycol (meth)acrylate, methoxydiethylene glycol (meth)acrylate, ethoxydiethylene glycol (meth)acrylate, butoxyethyl (meth)acrylate, butoxytriethylene glycol (meth)acrylate, 2-ethylhexylpolyethylene glycol (meth)acrylate, 4-hydroxybutyl (meth)acrylate, nonylphenylpolypropylene glycol (meth)acrylate, methoxydipropylene glycol (meth)acrylate, 2-hydroxyethyl (meth)acrylate, 2-hydroxypropyl (meth)acrylate, glycerol (meth)acrylate, polyethylene glycol (meth)acrylate, polypropylene glycol (meth)acrylate, epichlorohydrin-modified butyl (meth)acrylate, epichlorohydrin-modified phenoxy (meth)acrylate, N,N-dimethylaminoethyl (meth)acrylate, and N,N-diethylaminoethyl (meth)acrylate, but it is not limited thereto. Among these, as the monofunctional (meth)acrylate monomer, a monofunctional (meth)acrylate monomer having a hydroxyl group is preferably contained in the component (A). Specific examples of the monofunctional (meth)acrylate monomer having a hydroxyl group may include 2-hydroxyethyl (meth)acrylate and 2-hydroxypropyl (meth)acrylate, but it is not limited thereto. Among these, 2-hydroxypropyl (meth)acrylate is preferable and 2-hydroxypropyl acrylate is more preferable. As the monofunctional monomer having a hydroxyl group, a commercially available product may be used, and specific examples of the commercially available product may ACRYESTER (registered trademark) HO manufactured by Mitsubishi Rayon Co., Ltd., but it is not limited thereto.

As the (meth)acrylate monomer, a (meth)acrylate monomer having an acidic group may be used, and among these, it is preferable to use a monofunctional (meth)acrylate monomer having an acidic group as the monofunctional (meth)acrylate monomer. As the (meth)acrylate monomer having an acidic group, it is particularly preferable to use a carboxylic acid having a (meth)acryloyl group in the molecule, a phosphoric acid having a (meth)acryloyl group in the molecule, or the like, and it is more preferable to use a carboxylic acid having a (meth)acryloyloxy group in the molecule, a phosphoric acid having a (meth)acryloyloxy group in the molecule, or the like. Examples of the carboxylic acid having a (meth)acryloyl group in the molecule as the monofunctional (meth)acrylate monomer having an acidic group may include (meth)acrylic acid, 3-(meth)acryloyloxypropylsuccinic acid, 4-(meth)acryloyloxybutylsuccinic acid, 2-(meth)acryloyloxyethylmaleic acid, 3-(meth)acryloyloxypropylmaleic acid, 4-(meth)acryloyloxybutylmaleic acid, 2-(meth)acryloyloxyethylhexahydrophthalic acid, 3-(meth)acryloyloxypropylhexahydrophthalic acid, 4-(meth)acryloyloxybutylhexahydrophthalic acid, 2-(meth)acryloyloxyethylphthalic acid, 3-(meth)acryloyloxypropylphthalic acid, and 4-(meth)acryloyloxybutylphthalic acid, but it is not limited thereto. In addition, examples of the phosphoric acid having a (meth)acryloyl group in the molecule as the monofunctional (meth)acrylate monomer having an acidic group may include 2-ethylhexyl acid phosphate, 2-hydroxyethyl methacrylate acid phosphate, and dibutyl phosphate, but it is not limited thereto. Among these, as the monofunctional (meth)acrylate monomer having an acidic group, it is preferable to contain a carboxylic acid having a (meth)acryloyl group in the molecule and (meth)acrylic acid, and it is more preferable to contain methacrylic acid. As the (meth)acrylate monomer, it is preferable to contain a (meth)acrylate monomer having an acidic group for the purpose of improving the durability. Incidentally, as the (meth)acrylate monomer having an acidic group, a synthetic product or a commercially available product may be used.

Specific examples of the bifunctional (meth)acrylate monomer may include 1,3-butylene glycol di(meth)acrylate, 1,4-butylene glycol di(meth)acrylate, tricyclodecanedimethanol di(meth)acrylate, neopentyl glycol di(meth)acrylate, 1,6-hexane glycol di(meth)acrylate, ethylene glycol di(meth)acrylate, tetraethylene glycol di(meth)acrylate, polyethylene glycol di(meth)acrylate, propylene glycol di(meth)acrylate, tripropylene glycol di(meth)acrylate, ethylene oxide-modified neopentyl glycol di(meth)acrylate, propylene oxide-modified neopentyl glycol di(meth)acrylate, bisphenol A di(meth)acrylate, ethylene oxide-modified bisphenol A di(meth)acrylate, epichlorohydrin-modified bisphenol A di(meth)acrylate, ethylene oxide-modified bisphenol S di(meth)acrylate, neopentyl glycol-modified trimethylolpropane di(meth)acrylate, dicyclopentenyl di(meth)acrylate, ethylene oxide-modified dicyclopentenyl di(meth)acrylate, and diacryloyl isocyanurate, but it is not limited thereto.

Specific examples of the trifunctional (meth)acrylate monomer may include trimethylolpropane tri(meth)acrylate, trimethylolpropane tri(meth)acrylate, pentaerythritol tri(meth)acrylate, EO-modified trimethylolpropane tri(meth)acrylate, PO-modified trimethylolpropane tri(meth)acrylate, ECH-modified trimethylolpropane tri(meth)acrylate, ECH-modified glycerol tri(meth)acrylate, and tris(acryloyloxyethyl) isocyanurate, but it is not limited thereto.

Specific examples of the (meth)acrylamide monomer may include (meth)acrylamide, N-methyl(meth)acrylamide, N-ethyl(meth)acrylamide, N-propyl(meth)acrylamide, N-n-butyl(meth)acrylamide, N-tert-butyl(meth)acrylamide, N-butoxymethyl(meth)acrylamide, N-isopropyl(meth)acrylamide, N-methylol(meth)acrylamide, N,N-dimethyl(meth)acrylamide, (meth)acryloylmorpholine, N,N-diethyl(meth)acrylamide, N-methyl-N-ethyl(meth)acrylamide, and N-hydroxyethyl(meth)acrylamide, but it is not limited thereto. Among these, N,N-dimethyl(meth)acrylamide, (meth)acryloylmorpholine, or N,N-diethyl(meth)acrylamide is preferable, N,N-dimethyl(meth)acrylamide or (meth)acryloylmorpholine is more preferable, N,N-dimethylacrylamide or acryloylmorpholine is still more preferable, and N,N-dimethylacrylamide is particularly preferable from the viewpoint of improving the transmittance and durability. Although the clear reason is unknown, from the viewpoint of improving the durability, it is more preferable to contain a (meth)acrylamide monomer as the (meth)acrylate monomer. As the (meth)acrylamide monomer, a commercially available product may be used, DMAA (registered trademark), ACMO (registered trademark), DEAA (registered trademark), and the like manufactured by KJ Chemicals Corporation and the like are known as specific examples of the commercially available product, but it is not limited thereto.

A second embodiment which is a preferred embodiment of the present invention is the photocurable composition for nail or artificial nail according to the first embodiment in which the component (A) contains a (meth)acrylic oligomer and a (meth)acrylate monomer or a (meth)acrylamide monomer.

In a case in which the component (A) contains a (meth)acrylate oligomer and a (meth)acrylate monomer or a (meth)acrylamide monomer, the ratio (mass ratio) of the content of the (meth)acrylate oligomer to the total content of the (meth)acrylate monomer and the (meth)acrylamide monomer is preferably such that the content of the (meth)acrylate oligomer:the total content of the (meth)acrylate monomer and the (meth)acrylamide monomer=from 50:50 to 95:5, more preferably from 60:40 to 90:10, and still more preferably from 75:25 to 85:15. The durability is further improved as a (meth)acrylate oligomer is contained.

In addition, in a case in which the component (A) contains a (meth)acrylate monomer or a (meth)acrylamide monomer, the ratio (mass ratio) of the content of the (meth)acrylamide monomer to the content of the (meth)acrylate monomer is preferably such that the content of (meth)acrylamide monomer:the content of (meth)acrylate monomer=from 50:50 to 0:100, more preferably from 70:30 to 0:100, and still more preferably from 80:20 to 0:100.

A third embodiment of the present invention which is a preferred embodiment of the present invention is the photocurable composition for nail or artificial nail according to the second embodiment in which the (meth)acrylate monomer contains a (meth)acrylate monomer having an acidic group. At this time, those described above can be used as the (meth)acrylate monomer having an acidic group and a preferred (meth)acrylate monomer having an acidic group is also the same as the above.

In a case in which the component (A) contains a (meth)acrylate monomer or a (meth)acrylamide monomer, the ratio (mass ratio) of the content of the (meth)acrylate monomer having an acidic group to the total content of other (meth)acrylate monomers and the (meth)acrylamide monomer is more preferably such that the content of the (meth)acrylate monomer having an acid group:the total content of other (meth)acrylate monomers and the (meth)acrylamide monomer=more than 0: less than 100 to 20 or more: 80 or less and still more preferably from 5:95 to 20:80.

In a case in which the component (A) contains a (meth)acrylate monomer, the ratio (mass ratio) of the content of the (meth)acrylate monomer having an acidic group to the content of other (meth)acrylate monomers is preferably such that the content of the (meth)acrylate monomer having an acidic group: the content of other (meth)acrylate monomers=from 5:95 to 100:0, more preferably from 10:90 to 100:0, and still more preferably from 20:80 to 100:0.

In a case in which the component (A) contains a (meth)acrylate monomer, the (meth)acrylate monomer is preferably a monovalent (meth)acrylate monomer having an acidic group or a monovalent (meth)acrylate monomer having a hydroxyl group.

Incidentally, it is preferable to contain a (meth)acrylate oligomer having a urethane bond and a monofunctional (meth)acrylate monomer having an acidic group as the component (A), and it is more preferable to contain a (meth)acrylate oligomer having a urethane bond, a monofunctional (meth)acrylate monomer having an acidic group, and further a (meth)acrylamide monomer or a monofunctional (meth)acrylate monomer having a hydroxyl group as the component (A). In addition, it is particularly preferable to contain a (meth)acrylate oligomer having a urethane bond, a monofunctional (meth)acrylate monomer having an acidic group, and further a (meth)acrylamide monomer from the viewpoint of improving the durability and transmittance.

[Component (B)]

The component (B) is a component having a function as a plasticizer, and rosin or a rosin derivative is used. Here, rosin or a rosin derivative refers to a mixture of several kinds of isomers containing abietic acid as a main compound. Incidentally, abietic acid is a compound represented by the following formula 1 and is also conventionally referred to as rosin acid. As the component (B), a compound multimerized using rosin as a raw material, a derivative in which a side chain of rosin is chemically modified, and a compound obtained by hydrogenating rosin can also be contained. The softening point of the component (B) measured in conformity to JIS K5902: 2006 (ring and ball method) is preferably from 70° C. to 170° C., more preferably from 80° C. to 160° C., still more preferably from 80° C. to 140° C., yet still more preferably from 90° C. to 135° C., and particularly preferably from 95° C. to 130° C. from the viewpoint of improving the durability. Although the clear reason is unknown, it is possible to obtain a photocurable composition for nail or artificial nail which is colorless and transparent and exhibits improved soak off property by adding the component (B). In addition, the acid value of the component (B) measured in conformity to JIS K5902: 2006 is not particularly limited, but it is preferably from 1 to 300 mg KOH/g and more preferably from 2 to 250 mg KOH/g. Moreover, the color tone (Hazen) of the component (B) measured in conformity to the APHA method is not particularly limited, but it is preferably 300 or less.

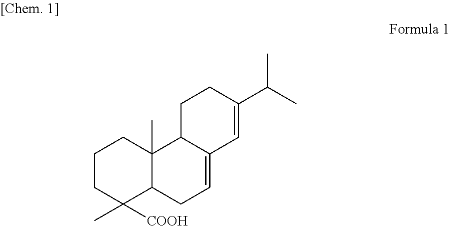 [SMILES: CC(C)C1=CC2=CCC3C(C)(C(=O)O)CCCC3(C)C2CC1]

As the component (B), a synthetic product or a commercially available product may be used, and specific examples of the commercially available product may include PINECRYSTAL (registered trademark) series KR-85, KR-612, KR-614, KE-100, KE-311, KE-359, KE-604, KR-120, KR-140, D-6011, KE-615-3, KM-1500, KR-50M, and the like manufactured by ARAKAWA CHEMICAL INDUSTRIES, LTD. and HARIESTER Series TF and S, NEOTALL (registered trademark) series G2, 101N, and 125HK, and HARITACK series 8L1A, ER95, SE10, PH, F85, F105, FK100, FK125, PCJ, and the like manufactured by Harima Chemicals Group, Inc., but it is not limited thereto. These may be used singly or two or more kinds thereof may be used concurrently.

The amount of the component (B) added is preferably 1 part by mass or more with respect to 100 parts by mass of the component (A). The soak off property is further improved in a case in which the amount of the component (B) added is 1 part by mass or more with respect to 100 parts by mass of the component (A). The amount of the component (B) added is more preferably 5 parts by mass or more and still more preferably 10 parts by mass or more with respect to 100 parts by mass of the component (A) from the same viewpoint. Meanwhile, the amount of the component (B) added is preferably 30 parts by mass or less with respect to 100 parts by mass of the component (A). The effect of maintaining durability such as adhesive force is further improved in a case in which the component (B) is 30 parts by mass or less. The amount of the component (B) added is more preferably 20 parts by mass or less and still more preferably 15 parts by mass or less with respect to 100 parts by mass of the component (A) from the same viewpoint. Consequently, a preferred amount of the component (B) added is, for example, 1 part by mass or more and 30 parts by mass or less with respect to 100 parts by mass of the component (A) and a more preferred amount of the component (B) added is, for example, 5 parts by mass or more and 20 parts by mass or less with respect to 100 parts by mass of the component (A).

[Component (C)]

The component (C) is a photoinitiator and is not limited as long as it is a radical photoinitiator which generates radical species by energy rays such as visible light, ultraviolet light, X-ray, and an electron beam.

Specific examples of the component (C) may include acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl) ketone, 1-hydroxycyclohexyl phenyl ketone, 2-methyl-2-morpholino(4-thiomethylphenyl)propane-1-one, 2-benzyl-2-dimethylamino-1-(4-morpholinophenyl)butanone, and a 2-hydroxy-2-methyl-1-[4-(1-methylvinyl)phenyl]propanone oligomer; benzoins such as benzoin, benzoin methyl ether, benzoin ethyl ether, benzoin isopropyl ether, and benzoin isobutyl ether; benzophenones such as benzophenone, methyl o-benzoylbenzoate, 4-phenylbenzophenone, 4-benzoyl-4′-methyl-diphenylsulfide, 3,3′,4,4′-tetra(t-butylperoxycarbonyl)benzophenone, 2,4,6-trimethylbenzophenone, 4-benzoyl-N,N-dimethyl-N-[2-(1-oxo-2-propenyloxy)ethyl]b enzenemethanaminium bromide, and (4-benzoylbenzyl)trimethylammonium chloride; thioxanthones such as 2-isopropylthioxanthone, 4-isopropylthioxanthone, 2,4-diethylthioxanthone, 2,4-dichlorothioxanthone, 1-chloro-4-propoxythioxanthone, and 2-(3-dimethylamino-2-hydroxy)-3,4-dimethyl-9H-thioxanthone-9-one mesochloride; and acylphosphine oxides (also referred to as an acylphosphine oxide-based photopolymerization initiator in the present specification) such as 2,4,6-trimethylbenzoyl-diphenyl-phosphine oxide and bis(2,4,6-trimethylbenzoyl)-phenylphosphine oxide, but it is not limited thereto. Among these, the component (C) preferably contains acetophenones. These may be used singly or plural components (C) of two or more kinds can be used in combination.

The amount of the component (C) added is preferably 0.1 parts by mass or more with respect to 100 parts by mass of the component (A). The effect of maintaining the photocurability is further improved in a case in which the amount of the component (C) added is 0.1 parts by mass or more with respect to 100 parts by mass of the component (A). The amount of the component (C) added is more preferably 0.14 parts by mass or more with respect to 100 parts by mass of the component (A) from the same viewpoint. Meanwhile, the amount of the component (C) added is preferably 20 parts by mass or less with respect to 100 parts by mass of the component (A). In a case in which the amount of the component (C) added is 20 parts by mass or less with respect to 100 parts by mass of the component (A), the effect of maintaining the storage stability is further improved as increasing viscosity at the time of storage is more favorably suppressed. The amount of the component (C) added is more preferably 10 parts by mass or less and still more preferably 5 parts by mass or less with respect to 100 parts by mass of the component (A) from the same viewpoint. Consequently, a preferred amount of the component (C) added is, for example, 0.1 parts by mass or more and 20 parts by mass or less with respect to 100 parts by mass of the component (A).

In addition, as the component (C), it is preferable to use a visible light type photoinitiator and a nonvisible light type photoinitiator from the viewpoint that the photocurability is further improved and coloration is less caused. In a case in which a visible light type photoinitiator and a nonvisible light type photoinitiator are used, it is preferable that the visible light type photoinitiator is contained at 70% by mass or less with respect to the entire component (C) (lower limit: 0% by mass). It is possible to suppress the occurrence of yellowing of the cured product by setting the visible light type photoinitiator to be in the above range. It is preferable that the visible light type photoinitiator is contained at 60% by mass or less with respect to the entire component (C) (lower limit: 0% by mass) from the same viewpoint. In addition, it is preferable that the visible light type photoinitiator is contained at 50% by mass or more with respect to the entire component (C). The photocurability is further improved by setting the visible light type photoinitiator to be in the above range.

Here, the visible light type photoinitiator is a photoinitiator which exhibits the highest light absorption in the visible light region, and examples thereof may include an acylphosphine oxide-based photopolymerization initiator mainly containing a phosphorus atom. Specific examples of the acylphosphine oxide-based photopolymerization initiator containing a phosphorus atom may include 2,4,6-trimethylbenzoyl-diphenyl-phosphine oxide and bis(2,4,6-trimethylbenzoyl)-phenylphosphine oxide, but it is not limited thereto.

[Coupling Material]

It is preferable to add a coupling agent to the photocurable composition for nail or artificial nail according to an embodiment of the present invention to an extent to which the properties of the present invention are not impaired. It is possible to further improve the adhesive property by adding a coupling agent.

The coupling agent is not particularly limited, but it is preferably a silane-based coupling agent. Examples of the silane-based coupling agent may include a silane-based coupling agent having both an epoxy group, a vinyl group, an acrylic group, or a methacrylic group and a hydrolyzable silane group, a polyorganosiloxane having a phenyl group and a hydrolyzable silyl group, and a polyorganosiloxane having an epoxy group and a hydrolyzable silyl group, but it is not limited thereto. Here, the hydrolyzable silyl group is not particularly limited, but examples thereof may include alkoxysilyl groups such as a methoxysilyl group and an ethoxysilyl group. Specific examples of the silane-based coupling agent may include allyltrimethoxysilane, vinyltrimethoxysilane, 3-glycidoxypropyltrimethoxysilane, 3-methacryloxypropyltrimethoxysilane, 3-acryloxypropyltrimethoxysilane, and 3-chloropropyltrimethoxysilane, but it is not limited thereto. Among these, 3-acryloxypropyltrimethoxysilane is preferable as the silane-based coupling agent. As the silane-based coupling material, a commercially available product can be used, and specific examples of the commercially available product may include KBM-1003, KBM-403, KBM-503, KBM-5103, and the like manufactured by Shin-Etsu Chemical Co., Ltd., but it is not limited thereto. These may be used singly or two or more kinds thereof may be used concurrently.

In the case of containing a silane-based coupling agent, the content thereof is preferably from 0.1 to 10 parts by mass, more preferably from 0.5 to 5 parts by mass, and still more preferably 1 to 3 parts by mass with respect to 100 parts by mass of the component (A).

[Filler]

It is preferable to appropriately add a filler such as an inorganic filler or an organic filler to the photocurable composition for nail or artificial nail according to an embodiment of the present invention to an extent to which the properties of the present invention are not impaired. By adding a filler, the curability and toughness can also be adjusted to be in more favorable ranges in addition to the viscosity and thixotropy.

Examples of the inorganic filler may include alumina, silica, and amorphous silica, but it is not limited thereto. Meanwhile, examples of the organic filler may include a styrene filler, a rubber filler, and a core-shell acrylic filler, but it is not limited thereto. Among these, an inorganic filler is preferable and amorphous silica is more preferable as the filler. As the filler, a synthetic product or a commercially available product may be used, and specific examples of the commercially available product may include FUSELEX (registered trademark) E-1 manufactured by Tatsumori Ltd. and AO-802 manufactured by Admatechs Company Limited, but it is not limited thereto. In addition, examples of amorphous silica may include AEROSIL series 200 (not treated), R972 (treated with dimethyldichlorosilane), R976 (treated with dimethyl dichlorosilane), RY200 (treated with dimethyl silicone), RX200 (treated with hexamethyldisilazane), R800 (treated with octylsilane), and the like manufactured by NIPPON AEROSIL CO., LTD., but it is not limited thereto. These may be used singly or two or more kinds thereof may be used concurrently.

In the case of containing a filler, the content thereof is preferably from 0.1 to 5 parts by mass, more preferably from 0.2 to 2 parts by mass, and still more preferably from 0.4 to 1 part by mass with respect to 100 parts by mass of the component (A).

[Other Additives]

In the present invention, other additives such as coloring agents such as a pigment and a dye, an antioxidant, a polymerization inhibitor, a defoaming agent, a leveling agent, and a rheology control agent may be blended in appropriate amounts to an extent to which the properties of the present invention are not impaired. By adding other additives including these depending on the purpose, it is possible to obtain a photocurable composition for nail or artificial nail which exhibits superior desired properties such as resin strength, adhesive force, workability, and storage stability or a cured product thereof.

<Method of Producing Photocurable Composition for Nail or Artificial Nail>

The method (adjusting method) of producing the photocurable composition for nail or artificial nail according to an embodiment of the present invention is not particular limited as long as it is possible to mix the component (A), the component (B), the component (C), and optionally usable components (a coupling agent, a filler, and other additives), and a known method can be used. Among these, a production method is preferable in which as the mixing order of the component (A), the component (B), and the component (C), first, the component (A) and the component (B) are weighed and put sequentially or simultaneously in a stirring vessel irrespective of the order and then stirred while conducting vacuum defoaming, thereafter, the component (C) is weighed and added to the stirring vessel, and the mixture is stirred.

<Base Coat Agent>

A fourth embodiment which is a preferred embodiment of the present invention is a base coat agent which contains the photocurable composition for nail or artificial nail according to any one of the first to third embodiments and can be soaked off. It is more preferable that the base coat agent which can be soaked off is the photocurable composition for nail or artificial nail according to any one of the first to third embodiments.

<Cured Product and Method of Producing Cured Product>

A fifth embodiment which is a preferred embodiment of the present invention is a cured product of the photocurable composition for nail or artificial nail according to any one of the first to third embodiments or the base coat agent according to the fourth embodiment. Here, it is preferable that the cured product is a base coat.

The method of producing the cured product (for example, base coat) of the photocurable composition for nail or artificial nail according to an embodiment of the present invention or the base coat agent according to an embodiment of the present invention is not particularly limited, and a known method can be used. Among these methods, a method is preferable in which a photocurable composition for nail or artificial nail or abase coat agent is cured using energy rays. In other words, a sixth embodiment which is a preferred embodiment of the present invention is a method of producing a cured product in which the photocurable composition for nail or artificial nail according to any one of the first to third embodiments or the base coat agent according to the fourth embodiment is cured by energy rays.

A preferred example of such a method is described below, but the method of producing a cured product is not limited to this method. First, the surface of a human nail or artificial nail (particularly preferably a human nail) is sanded using a file or the like before the photocurable composition for nail or artificial nail or the base coat agent is applied. Subsequently, the dust, oil, moisture, and the like on the nail are removed using a solvent for nail exclusive use containing ethanol as a main component. Thereafter, the photocurable composition for nail or artificial nail according to the present invention or a base coat agent containing this is applied on the nail. Here, at the time of applying, it is preferable to form a coating film having a thickness of 100 μm or more and 300 μm or less in an uncured state (wet state) using a brush, a paintbrush, or the like. In addition, at the time of applying, a primer may be used in advance. As an irradiation apparatus for curing, it is preferable to use a commercially available UV lamp or LED lamp. The irradiation time is preferably 10 seconds or longer and 120 seconds or shorter, more preferably 15 seconds or longer and 120 seconds or shorter, and it is more preferably 20 seconds or longer and 70 seconds or shorter and particularly preferably 20 seconds or longer and 60 seconds or shorter when the influence on the finger is taken into consideration. Here, when using a UV lamp, it is preferable to use a condition that the irradiation time is 15 seconds or longer and 120 seconds or shorter. The integrated light quantity is preferably from 5 to 60 kJ/m². Incidentally, at the time of curing, energy irradiation may be conducted plural times if necessary.

<Soaking Off Method>

The method of soaking off the cured product (for example, base coat) of the photocurable composition for nail or artificial nail according to an embodiment of the present invention or the base coat agent according to an embodiment of the present invention is not particularly limited, and a known method can be used. Among these methods, it is preferable to use a method in which the cured product is soaked off (peeled off) using warm water or a solvent. In other words, a seventh embodiment which is a preferred embodiment of the present invention is a peeling method which includes peeling off the cured product according to the fifth embodiment or a cured product produced by the method according to the sixth embodiment which is disposed on the substrate using warm water or a solvent. Here, warm water represents water at a temperature higher than normal temperature (25° C.), and the temperature is preferably higher than normal temperature and 40° C. or lower and more preferably 35° C. or higher and 40° C. or lower. The reason why this range is preferable is because the soak off property becomes favorable by setting the temperature to be higher than normal temperature and a temperature of 40° C. or lower is preferable when the influence on the finger is taken into consideration. Incidentally, in the present specification, formation on the substrate may be formation directly on the surface of the substrate or formation on the outermost surface of a single layer or a plurality of other layers formed on the surface side of the substrate. Here, in an embodiment of the present invention, the substrate is preferably a human nail or an artificial nail and more preferably a human nail, and thus the soaking off method is more preferably a peeling method in which the cured product according to the fifth embodiment or a cured product produced by the method according to the sixth embodiment formed on a human nail or an artificial nail (particularly preferably a human nail) is peeled off using warm water or a solvent. Moreover, the soaking off method is still more preferably a peeling method in which the cured product according to the fifth embodiment or a cured product produced by the method according to the sixth embodiment formed directly on the surface of a human nail or an artificial nail (particularly preferably a human nail) is peeled off using warm water or a solvent.

It is preferable to use a solvent as warm water or a solvent to be used at the time of soaking off. As a preferred solvent, acetone may be mentioned from the viewpoint of soak off property, but it is not particularly limited thereto.

A preferred example of such a method is described below, but the peeling method is not limited to this method. First, the top coat and the color coat are removed through sanding using a file or the like in order to soak off the cured product of a photocurable composition for nail or artificial nail or a base coat agent. Subsequently, cotton impregnated with acetone is wound around a human nail or an artificial nail (particularly preferably a human nail) and left in a state of being wrapped with an aluminum foil. Thereafter, the cured product not firmly fixed is lightly scraped off as the cured product becomes a state where it is not firmly fixed after a certain period of time elapses.

<Preferred Use Method>

Here, an eighth embodiment which is a preferred embodiment of the present invention is a method of covering a human nail or an artificial nail which includes applying the photocurable composition for nail or artificial nail according to any one of the first to third embodiments or the base coat agent according to the fourth embodiment on a human nail or an artificial nail (particularly preferably a human nail) to form a coating film and then irradiating the coating film with an energy ray for curing. Incidentally, in the present specification, applying on a human nail or an artificial nail may be applying directly on the surface of a human nail or artificial nail or applying on the outermost surface of a single layer or a plurality of other layers formed on the surface of a human nail or artificial nail. Moreover, the method of using the photocurable composition for nail or artificial nail according to an embodiment of the present invention or the base coating agent according to an embodiment of the present invention is more preferably a method of covering a human nail or an artificial nail in which the photocurable composition for nail or artificial nail according to any one of the first to third embodiments or the base coat agent according to the fourth embodiment is directly applied on the surface of a human nail or artificial nail (particularly preferably a human nail) to forma coating film and then the coating film is cured by being irradiated with energy rays. In addition, a ninth embodiment which is a still preferred embodiment of the present invention is a use method which includes covering a human nail or an artificial nail (particularly preferably a human nail) by the method of the eighth embodiment and then peeling off the cured product which is a covering layer using warm water or a solvent. As the conditions of warm water, formation conditions of coating film, and irradiation conditions of energy rays in these methods, the same conditions as those for the cured product and the method of producing a cured product described above can be used. However, the method of using the photocurable composition for nail or artificial nail according to an embodiment of the present invention or the base coat agent according to an embodiment of the present invention is not limited to these methods, and the method of using cured products of these is also not limited to these methods.

### EXAMPLES

Next, the present invention will be described in more detail with reference to Examples, but the present invention is not limited only to these Examples.

<Preparation of Photocurable Composition for Nail or Artificial Nail>

### Examples 1 to 7 and Comparative Examples 1 to 7

In order to prepare a photocurable composition for nail or artificial nail (hereinafter also simply referred to as a composition), the following components were prepared.

<Component (A): Compound Having (Meth)Acryl Group>
Urethane-modified acrylate oligomer (bifunctional, weight average molecular weight: 4500) (UF-8001G manufactured by KYOEISHA CHEMICAL CO., LTD., denoted as “UF8001G” in the following Table 1 and Table 2)
N,N-dimethylacrylamide (DMAA (registered trademark) manufactured by KJ Chemicals Corporation)
Acryloylmorpholine (ACMO (registered trademark) manufactured by KJ Chemicals Corporation)
2-Hydroxyethyl acrylate (ACRYESTER (registered trademark) HO manufactured by Mitsubishi Rayon Co., Ltd., denoted as “HO” in the following Table 1 and Table 2)
Methacrylic acid (METHACRYLIC ACID manufactured by Mitsubishi Rayon Co., Ltd.).

<Component (B): Rosin or Rosin Derivative>
Rosin ester (solid at 25° C.) (softening point: 95° C.) (PINECRYSTAL (registered trademark) KE-311 manufactured by ARAKAWA CHEMICAL INDUSTRIES, LTD., denoted as “KE-311” in the following Table 1 and Table 2)
Rosin ester (solid at 25° C.) (softening point: 100° C.) (PINECRYSTAL (registered trademark) KE-359 manufactured by ARAKAWA CHEMICAL INDUSTRIES, LTD., denoted as “KE-359” in the following Table 1 and Table 2)
Acid modified rosin (solid at 25° C.) (softening point: 130° C.) (PINECRYSTAL (registered trademark) KE-604 manufactured by ARAKAWA CHEMICAL INDUSTRIES, LTD., denoted as “KE-604” in the following Table 1 and Table 2)

<(B′) Component: Plasticizer Other than Component (B)>
Alkylphenol resin (solid at 25° C.) (TAMANOL (registered trademark) 200N manufactured by ARAKAWA CHEMICAL INDUSTRIES, LTD., denoted as “200N” in the following Table 1 and Table 2)
Maleic anhydride-modified polybutadiene (solid at 25° C.) (POLYVEST (registered trademark) MA75 manufactured by Evonik Industries AG, denoted as “MA75” in the following Table 1 and Table 2)
Polycarbonate polyol (liquid at 25° C.) (Kuraray (registered trademark) C-2050 manufactured by KURARAY CO., LTD., denoted as “C-2050” in the following Table 1 and Table 2)
Polyoxypropylene glyceryl ether (liquid at 25° C.) (ADEKA POLYETHER G-4000 manufactured by ADEKA Corporation, denoted as “G-4000” in the following Table 1 and Table 2)
Ethylenediamine-ethylene oxide modified product (liquid at 25° C.) (ADEKA POLYETHER BM-34 manufactured by ADEKA Corporation, denoted as “BM-34” in the following Table 1 and Table 2)
Dipropylene glycol dibenzoate (liquid at 25° C.) (ADK CIZER (registered trademark) PN-6120 manufactured by ADEKA Corporation, denoted as “PN-6120” in the following Table 1 and Table 2).

<Component (C): Photoinitiator>
2,4,6-Trimethylbenzoyl-diphenyl-phosphine oxide (visible light type photoinitiator) (LUCIRIN (registered trademark) TPO manufactured by BASF SE, denoted as “TPO” in the following Table 1 and Table 2)
1-Hydroxycyclohexyl phenyl ketone (invisible light type photoinitiator) (Suncure 84 manufactured by Chemark Chemical Co., Ltd., denoted as “84” in the following Table 1 and Table 2).

<Coupling Agent>
3-Acryloxypropyltrimethoxysilane (KBM-5103 manufactured by Shin-Etsu Chemical Co., Ltd.).

<Filler>
Non-treated fumed silica (BET specific surface area: 200 m²/g) (AEROSIL (registered trademark) 200 manufactured by NIPPON AEROSIL CO., LTD., denoted as “200” in the following Table 1 and Table 2).

For the preparation of a composition, first, the component (A), the component (B) (or the component (B′)), and the coupling agent were weighed and put into a stirring vessel and then stirred for 30 minutes while conducting vacuum defoaming. At this time, the stirring time was extended until the component (B) (or the component (B′)) was dissolved in a case in which the component (B) (or the component (B′)) was solid. Thereafter, the filler was weighed and added to the stirring vessel and the mixture was further stirred for 30 minutes while conducting vacuum defoaming. Finally, the component (C) was weighed and added to a stirring vessel and the mixture was stirred for 30 minutes. Detailed amounts of the respective compositions prepared were as presented in the following Table 1. Incidentally, in the following Table 1, the numerical values are all expressed in parts by mass.

TABLE 1

| | Raw | | | | | | | |
|---|---|---|---|---|---|---|---|---|
| Components | materials | Example 1 | Example 2 | Example 3 | Example 4 | Example 5 | Example 6 | Example 7 |
| Component | UF-8001G | 75 | 75 | 75 | 75 | 75 | 75 | 75 |
| (A) | DMAA | 20 | 20 | 20 | | | | |
| | ACMO | | | | 20 | 20 | | |
| | HO | | | | | | 20 | 20 |
| | Methacrylic | 5 | 5 | 5 | 5 | 5 | 5 | 5 |
| | acid | | | | | | | |
| Component | KE-311 | 15 | | | 15 | | 15 | |
| (B) | KE-359 | | 15 | | | 15 | | 15 |
| | KE-604 | | | 15 | | | | |
| Component | 200N | | | | | | | |
| (B′) | MA75 | | | | | | | |
| | C-2050 | | | | | | | |
| | G-4000 | | | | | | | |
| | BM-34 | | | | | | | |
| | PN-6120 | | | | | | | |
| Component | TPO | 3.0 | 3.0 | 3.0 | 3.0 | 3.0 | 3.0 | 3.0 |
| (C) | 84 | 2.0 | 2.0 | 2.0 | 2.0 | 2.0 | 2.0 | 2.0 |
| Coupling | KBM-5103 | 1.0 | 1.0 | 1.0 | 1.0 | 1.0 | 1.0 | 1.0 |
| agent | | | | | | | | |
| Filler | 200 | 0.5 | 0.5 | 0.5 | 0.5 | 0.5 | 0.5 | 0.5 |

| | | | | | | | |
|---|---|---|---|---|---|---|---|
| Sum | 121.5 | 121.5 | 121.5 | 121.5 | 121.5 | 121.5 | 121.5 |

| | Raw | Comparative | Comparative | Comparative | Comparative | Comparative | Comparative | Comparative |
|---|---|---|---|---|---|---|---|---|
| Components | materials | Example 1 | Example 2 | Example 3 | Example 4 | Example 5 | Example 6 | Example 7 |
| Component | UF-8001G | 75 | 75 | 75 | 75 | 75 | 75 | 75 |
| (A) | DMAA | 20 | 20 | 20 | 20 | 20 | 20 | 20 |
| | ACMO | | | | | | | |
| | HO | | | | | | | |
| | Methacrylic | 5 | 5 | 5 | 5 | 5 | 5 | 5 |
| | acid | | | | | | | |
| Component | KE-311 | | | | | | | |
| (B) | KE-359 | | | | | | | |
| | KE-604 | | | | | | | |
| Component | 200N | 15 | | | | | | |
| (B′) | MA75 | | 15 | | | | | |
| | C-2050 | | | 15 | | | | |
| | G-4000 | | | | 15 | | | |
| | BM-34 | | | | | 15 | | |
| | PN-6120 | | | | | | 15 | |
| Component | TPO | 3.0 | 3.0 | 3.0 | 3.0 | 3.0 | 3.0 | 3.0 |
| (C) | 84 | 2.0 | 2.0 | 2.0 | 2.0 | 2.0 | 2.0 | 2.0 |
| Coupling | KBM-5103 | 1.0 | 1.0 | 1.0 | 1.0 | 1.0 | 1.0 | 1.0 |
| agent | | | | | | | | |
| Filler | 200 | 0.5 | 0.5 | 0.5 | 0.5 | 0.5 | 0.5 | 0.5 |

| | | | | | | | |
|---|---|---|---|---|---|---|---|
| Sum | 121.5 | 121.5 | 121.5 | 121.5 | 121.5 | 121.5 | 106.5 |

For Examples 1 to 7 and Comparative Examples 1 to 7, the measurement of viscosity, confirmation of surface curability, measurement of transmittance, measurement of turbidity, confirmation of soak off property, and test of durability were conducted according to the methods described later. The results thereof are summarized in the following Table 2.

[Measurement of Viscosity]

The composition was sampled by 0.5 ml and discharged into a measuring cup. Thereafter, the viscosity of the composition was measured using an EHD type viscometer (manufactured by TOKI SANGYO CO., LTD.) under the following conditions. The result thereof was adopted as the “viscosity (Pa·s)”. The viscosity is preferably 30 Pa·s or less and more preferably 25 Pa·s or less from the viewpoint of flowability and the like when handling at the time of application is taken into consideration. In addition, the viscosity is preferably 5 Pa·s or more from the viewpoint of flowability.

(Measurement Conditions)

Cone rotor: 3°×R14

Rotational speed: 10 rpm

Measurement time: 3 minutes

Measurement temperature: 25° C. (temperature controlled by constant temperature bath)

[Confirmation of Surface Curability]

The composition was applied on an acrylic plate having a thickness of 2.0 mm×a width of 25 mm×a length of 100 mm using a paintbrush so as to have a thickness of 300 μm in a wet state. Subsequently, the composition was cured by being irradiated using a nail UV lamp (rated voltage: 100 to 110 V 50 to 60 Hz, power consumption: 36 W, wavelength: 350 to 400 nm) for 60 seconds. The cured state at that time was judged according to the following evaluation criteria, and the judgement was adopted as the “surface curability (UV lamp)”. In addition, the composition was cured by being irradiated using a nail LED lamp (rated voltage: 240 V 50 to 60 Hz, power consumption: 30 W, wavelength: 400 to 410 nm) for 10 seconds in the same manner. The cured state at that time was visually judged according to the following evaluation criteria, and the judgement was adopted as the “surface curability (LED lamp)”. The surface curability is preferably such that the surface is cured independently of the kind of lamp and both the “surface curability (UV lamp)” and the “surface curability (LED lamp)” are “0”.

(Evaluation Criteria)

◯: bleeding of components does not occur on surface

X: bleeding of components occurs on surface.

[Measurement of Transmittance]

A SUS 304 spacer having a thickness of 1 mm was placed on both ends of an alkali-free glass plate having a length of 50 mm×a width of 25 mm×a thickness of 0.7 mm, and 1 g of the composition was applied between the spacers. Subsequently, another alkali-free glass plate was placed so that the composition was sandwiched therebetween and bubbles did not enter therebetween, thereby fabricating a test piece. At this time, the spilt over portion of the composition was wiped off since the spilt over portion flowed out. Thereafter, the test piece thus fabricated was irradiated two times using a nail UV lamp (rated voltage: AC 100 V 50 to 60 Hz, power consumption: 36 W, wavelength: 350 to 400 nm) for 60 seconds to cure the composition. Incidentally, it was set n=1 in the fabrication of test piece. Subsequently, the test piece containing the composition after being cured was subjected to the measurement using an ultraviolet and visible spectrophotometer UV-2450 manufactured by Shimadzu Corporation. The measurement was conducted two times, and the average value thereof was calculated. The measurement range and conditions were set as follows, wavelength range in measurement: 300 to 800 nm, scan speed: high speed, sampling pitch: 1.0, and slit width: 5.0 nm. The measurement of baseline was conducted using an alkali-free glass plate used for the test piece. The “transmittances (o)” at wavelengths of 450 nm, 420 nm, and 400 nm were measured in this manner. It is preferable that the transmittances at the respective wavelengths are respectively 95% or more, 90% or more, and 30% or more (upper limit: 100%) when the appearance is taken into consideration.

[Measurement of Turbidity]

A SUS 304 spacer having a thickness of 1 mm was placed on both corners of an alkali-free glass plate having a length of 50 mm×a width of 25 mm×a thickness of 0.7 mm, and 1 g of the composition was applied on the glass plate. Subsequently, the other glass plate was gently placed thereon so that air bubbles did not enter the composition, thereby fabricating a test piece. At this time, the spilt over portion of the composition was wiped off since the spilt over portion flowed out. Thereafter, the test piece thus fabricated was irradiated two times using a nail UV lamp (rated voltage: AC 100 V 50 to 60 Hz, power consumption: 36 W, wavelength: 350 to 400 nm) for 60 seconds to cure the composition. Incidentally, it was set n=1 in the fabrication of test piece. Subsequently, the test piece containing the composition after being cured was subjected to the measurement using a haze meter NDH 2000 manufactured by Nippon Denshoku Industries Co., Ltd., and the result thereof was adopted as the “turbidity (%)”. The turbidity was measured three times, and the average value thereof was adopted. It is preferable that the “turbidity” is less than 1% (lower limit: 0%) from the viewpoint of appearance.

[Confirmation of Soak Off Property]

The composition was poured into a mold having a width of 30 mm×a length of 90 mm×a thickness of 0.5 mm and cured by being irradiated at an integrated light quantity of 30 kJ/m² by a belt conveyor type irradiator using a high pressure mercury lamp, and then the cured product of the composition was released from the mold and further irradiated at an integrated light quantity of 30 kJ/m². Subsequently, the cured product was left to stand for 2 hours after being cured. Thereafter, six sheets of the cured products after being left to stand were stacked and subjected to the measurement of hardness. Here, first, the cured product was quickly pressed at a force of 10 N without applying an impact while keeping the pressurizing surface of the D-type durometer (hardness tester) parallel to the sheet-like cured product to bring the pressurizing surface into close contact with the cured product. Thereafter, the maximum value at the time of measurement was read, and the maximum value was adopted as the “hardness before immersion (no unit)”. Incidentally, the details of the measurement conformed to JIS K6253-3: 2012. Subsequently, the cured product subjected to the measurement of hardness before immersion was immersed in acetone for 10 minutes, and the hardness thereof was measured again in the same manner as in the measurement of hardness before immersion, and the result thereof was adopted as the “hardness after immersion (no unit)”. The value calculated by ((hardness before immersion−hardness after immersion)/(hardness before immersion)×100” was adopted as the “soak off property (%)”. It is preferable that the “soak off property” is preferably 70% or more and 100% or less from the viewpoint of leading the cured product to a state of being easily peeled off in a shorter time after being immersed in a solvent.

[Test of Durability]

The dust and oil were removed from the nail using a solvent for nail exclusive use (containing ethanol as a main component) after sanding was conducted. Subsequently, the composition as a base coat agent was applied on the nail so as to have a thickness of 300 μm in a wet state. Applying was conducted using a paintbrush. Thereafter, the composition was cured by being irradiated using a nail LED lamp (rated voltage: 240 V 50-60 Hz, power consumption: 30 W, wavelength: 400 to 410 nm) for 10 seconds to obtain a base coat. Subsequently, a color coat and a top coat were sequentially formed on the surface of the base coat by conducting curing under the same conditions by the same method. Here, Super Color EX (color: pastel peach) manufactured by PREGEL was used for the color coat and VL-00 manufactured by VETRO was used for the top coat, respectively. As the evaluation, the number of fingernails which did not undergo peeling off in 3 weeks after application with respect to the fingernails (10 nails) of the hands of one human was adopted as the “durability (nails/10 nails)”. Here, peeling off included both peeling off from the entire surface and peeling off only from the end portion. It is preferable that the “durability” is preferably 5 or more when the practicality is taken into consideration.

TABLE 2

| Items of test | | Example 1 | Example 2 | Example 3 | Example 4 | Example 5 | Example 6 | Example 7 |
|---|---|---|---|---|---|---|---|---|
| Surface curability (UV | | ◯ | ◯ | ◯ | ◯ | ◯ | ◯ | ◯ |
| lamp) | | | | | | | | |
| Surface curability (LED | | ◯ | ◯ | ◯ | ◯ | ◯ | ◯ | ◯ |
| lamp) | | | | | | | | |
| Transmittance | 450 nm | 99.7 | 99.3 | 99.4 | 99.4 | 99.3 | 99.3 | 99.3 |
| | 420 nm | 97.4 | 96.8 | 96.7 | 97.1 | 96.9 | 96.4 | 96.9 |
| | 400 nm | 41.7 | 38.9 | 35.4 | 37.3 | 38.3 | 35.5 | 38.5 |

| | | | | | | | |
|---|---|---|---|---|---|---|---|
| Turbidity | 0.53 | 0.5 | 0.02 | 0.45 | 0.7 | 0.55 | 0.6 |
| Soak off property | 81 | 79 | 75 | 77 | 87 | 83 | 79 |
| Viscosity | 23 | 24 | 24 | 22 | 23 | 19 | 20 |
| Durability | 7 | 6 | 6 | 7 | 6 | 5 | 5 |

| | | Comparative | Comparative | Comparative | Comparative | Comparative | Comparative | Comparative |
|---|---|---|---|---|---|---|---|---|
| Items of test | | Example 1 | Example 2 | Example 3 | Example 4 | Example 5 | Example 6 | Example 7 |
| Surface curability (UV | | ◯ | ◯ | ◯ | X | X | X | ◯ |
| lamp) | | | | | | | | |
| Surface curability (LED | | ◯ | ◯ | ◯ | X | X | X | ◯ |
| lamp) | | | | | | | | |
| Transmittance | 450 nm | 92.1 | 44.3 | 34.1 | 97.0 | 97.1 | 99.0 | 98.5 |
| | 420 nm | 84.5 | 35.7 | 26.7 | 94.2 | 92.9 | 96.2 | 92.9 |
| | 400 nm | 30.7 | 18.1 | 11.5 | 48.0 | 38.8 | 45.6 | 10.0 |

| | | | | | | | |
|---|---|---|---|---|---|---|---|
| Turbidity | 0.24 | 37.2 | 46.8 | 1.1 | 0.5 | 0.43 | 0.45 |
| Soak off property | 72 | 71 | 83 | 85 | 71 | 87 | 40 |
| Viscosity | 25 | 32 | 33 | 21 | 16 | 17 | 23 |
| Durability | 6 | 6 | 6 | 6 | 6 | 5 | 5 |

In the compositions according to Examples, rosin or a rosin derivative was added as the component (B) and thus favorable results were obtained for all of the surface curability, transmittance, turbidity, soak off property, viscosity, and durability.

On the other hand, an alkylphenol resin was added instead of the component (B) in Comparative Example 1 and maleic anhydride-modified polybutadiene was added instead of the component (B) in Comparative Example 2, but the transmittance was low at 450 nm and 420 nm in the composition according to Comparative Example 1 and the transmittance was low and the turbidity was high in the composition according to Comparative Example 2. Hence, Comparative Examples 1 and 2 are inferior to Examples in appearance.

In addition, in Comparative Examples 3 to 6, a plasticizer which was liquid at 25° C. was added instead of the component (B) in order to improve the soak off property, but the turbidity was remarkably high in the composition according to Comparative Example 3 and the surface curability was low in the compositions according to Comparative Examples 4 to 6.

Moreover, a component corresponding to the component (B) was not contained in Comparative Example 7, and thus the soak off property was low in the composition according to Comparative Example 7.

### INDUSTRIAL APPLICABILITY

The photocurable composition for nail or artificial nail according to the present invention can be used as a base coat agent capable of forming a base coat on which a top coat and a nail color can be stably applied in accordance with the application in the field of nails, and soaking off of the cured product thereof can also be promptly conducted. The photocurable composition for nail or artificial nail according to the present invention is a photocurable composition suitable for forming a base coat which is transparent and colorless and is not turbid on a nail or an artificial nail, and thus it can be widely used in the field of nails.

This application is based upon and claims the benefit of priority of the prior Japanese Patent Application No. 2015-219338 filed on Nov. 9, 2015, the entire contents of which are incorporated herein by reference.

1-9. (canceled)

10. A photocurable composition for a nail or artificial nail comprising:
component (A): a compound having a (meth)acryloyl group;
component (B): rosin or a rosin derivative; and
component (C): a photoinitiator.

11. The photocurable composition for a nail or artificial nail according to claim 10, wherein the component (A) comprises a (meth)acrylate oligomer and a (meth)acrylate monomer or a (meth)acrylamide monomer.

12. The photocurable composition for a nail or artificial nail according to claim 11, wherein the (meth)acrylate monomer comprises a (meth)acrylate monomer having an acidic group.

13. A base coat agent comprising the photocurable composition for a nail or artificial nail according to claim 10, wherein the base coat agent can be soaked off.

14. A cured product of the photocurable composition for a nail or artificial nail according to claim 10.

15. A method of producing a cured product, the method comprising curing the photocurable composition for a nail or artificial nail according to claim 10.

16. A peeling method comprising peeling off the cured product according to claim 14 which is disposed on a substrate from the substrate using warm water or a solvent.

17. A method of covering a human nail or an artificial nail, the method comprising applying the photocurable composition for nail or artificial nail according to claim 10 on a human nail or an artificial nail to form a coating film and then irradiating the coating film with an energy ray for curing.

18. A use method comprising covering a human nail or an artificial nail by the covering method according to claim 17 and then peeling off a cured product of a covering layer using warm water or a solvent.
